(19) United States
(12) Patent Application Publication
(10) Pub. No.: US 2018/0132826 A1
(43) Pub. Date: May 17, 2018

(54) ULTRASONIC PROBE

(71) Applicant: Toshiba Medical Systems Corporation, Otawara-shi (JP)

(72) Inventors: Yuya Nagasaki, Nasushiobara (JP); Kiri Hyomoto, Otawara (JP); Koichi Shibamoto, Otawara (JP); Kentaro Tsuzuki, Nasushiobara (JP)

(73) Assignee: Toshiba Medical Systems Corporation, Otawara-shi (JP)

(21) Appl. No.: 15/801,859

(22) Filed: Nov. 2, 2017

(30) Foreign Application Priority Data
Nov. 11, 2016 (JP) 2016-220277
Oct. 6, 2017 (JP) 2017-195950

(51) Int. Cl. A61B 8/00; B06B 1/06; A61B 8/12; G01N 29/24

(52) U.S. Cl. CPC A61B 8/4494; B06B 1/0622; H01L 41/338; G01N 29/2437; B06B 2201/76; A61B 8/12

(57) ABSTRACT

According to one embodiment, an ultrasonic probe includes a plurality of piezoelectric elements, a substrate, an intermediate layer and a backing member. A plurality of piezoelectric elements are arranged at a predetermined pitch. A substrate is arranged on a back surface of the plurality of piezoelectric elements and includes signal lines for signal transmission with the plurality of piezoelectric elements and a wiring pattern for extracting a signal outside of the probe. An intermediate layer is arranged on a back surface of the substrate and in which a plurality of layer members are arranged in an array direction of the piezoelectric elements at the predetermined pitch. A backing member is arranged on a back surface of the intermediate layer.

### CROSS-REFERENCE TO RELATED APPLICATIONS

This application is based upon and claims the benefit of priority from the prior Japanese Patent Applications No. 2016-220277, filed Nov. 11, 2016 and No. 2017-195950, filed Oct. 6, 2017, the entire contents of all of which are incorporated herein by reference.

### FIELD

Embodiments described herein relate generally to an ultrasonic probe.

### BACKGROUND

An ultrasonic diagnostic apparatus transmits an ultrasonic signal to an object (patient), and receives a reflection signal (echo signal) from the inside of the object to obtain an image of the inside of the object, and an electronically-operated; array-type ultrasonic probe having a function of transmitting/receiving an ultrasonic signal is mainly used with the apparatus.

A common ultrasonic probe has a plurality of piezoelectric bodies arranged in an array and a flexible print circuit board (hereinafter, FPC) for extracting electrodes from the piezoelectric bodies, and a backing member. Generally, piezoelectric bodies are diced in a direction of acoustic radiation and separated into a plurality of elements to form a plurality of piezoelectric elements arranged in an array.

However, a high wiring density of signal lines in an FPC is demanded for an ultrasonic probe in which a pitch between piezoelectric elements is narrow, such as an octagonal-shaped bar hole probe used for brain surgery or a so-called 1.5-dimensional array probe. To meet such demands, an FPC may have a two-layer structure consisting of a first layer which is connected to piezoelectric bodies and a second layer for extracting a wiring pattern to the outside of the FPC.

In such a two-layer structure, at least the second layer is desirably continuous in the array direction, in other words, the second layer is not cut, so that the wiring pattern is not cut.

However, since the continuous second layer is not cut, a backing member has no cutting (a groove or a gap) formed as a result of dicing, and this causes degradation in sensitivity of transmitting/receiving ultrasonic waves.

### BRIEF DESCRIPTION OF THE DRAWING

FIG. 1 is a drawing showing an example of an outer appearance of an ultrasonic probe according to an embodiment.

FIG. 2 is a drawing showing an inner structure of an ultrasonic probe according to the embodiment.

FIG. 3A is a drawing illustrating an example of a wiring pattern for a first layer of an FPC.

FIG. 3B is a drawing illustrating an example of a wiring pattern for a second layer of an FPC.

FIG. 4 is a drawing showing a computation model for the inner structure of the ultrasonic probe for simulation using a finite element method.

FIG. 5 is a drawing showing a simulation result based on the computation model shown in FIG. 4.

FIG. 6 is a drawing showing an inner structure of an ultrasonic probe according to a first modification.

FIG. 7 is a drawing showing an inner structure of an ultrasonic probe according to a second modification.

FIG. 8 is a drawing showing an example of a wiring pattern for an FPC in an ultrasonic probe of a 1.5-dimensional array-type.

FIG. 9 is a drawing showing an inner structure of an ultrasonic probe as a conventional example.

FIG. 10 is a drawing showing an inner structure of an ultrasonic probe as a conventional example.

FIG. 11 is a drawing showing an inner structure of an ultrasonic probe according to a third modification.

FIG. 12 is a drawing showing an inner structure of an ultrasonic probe according to a fourth modification.

### DETAILED DESCRIPTION

In general, according to one embodiment, an ultrasonic probe includes a plurality of piezoelectric elements, a substrate, an intermediate layer and a backing member. A plurality of piezoelectric elements are arranged at a predetermined pitch. A substrate is arranged on a back surface of the plurality of piezoelectric elements and includes signal lines for signal transmission with the plurality of piezoelectric elements and a wiring pattern for extracting a signal outside of the probe. An intermediate layer is arranged on a back surface of the substrate and in which a plurality of layer members are arranged in an array direction of the piezoelectric elements at the predetermined pitch. A backing member is arranged on a back surface of the intermediate layer.

An ultrasonic probe according to the present embodiment will be described with reference to the accompanying drawings. In the embodiment described below, elements assigned with the same reference signs perform the same operations, and redundant descriptions thereof will be omitted as appropriate.

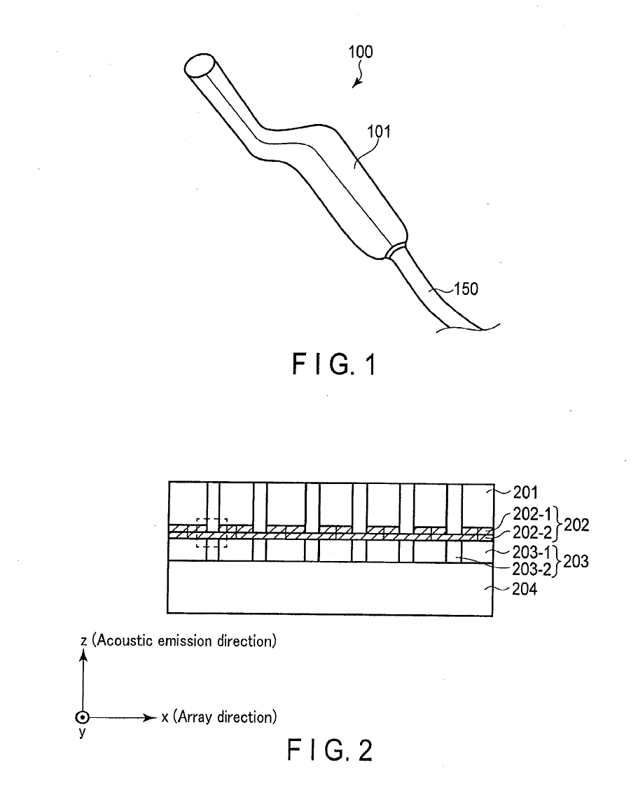

An example of the outer appearance of the ultrasonic probe 100 according to the present embodiment is shown in FIG. 1. A bar hole probe having an octagonal shape is assumed in the present embodiment.

An ultrasonic probe is coupled to an ultrasonic diagnostic apparatus main body (not shown) by a cable 150. Hereinafter, for convenience of explanation, in the ultrasonic probe 100, the side where an ultrasonic wave is transmitted and received between the ultrasonic probe 100 and a subject may be called a distal side, and the side connected to the cable 150 may be called a proximal side.

A housing 101 of the ultrasonic probe 100 is made of a typical material for a housing of a common ultrasonic probe, for example, a resin. The housing 101 is formed to have a sealing structure for water resistance, which allows the housing to withstand cleaning, etc.

One end of the cable 150 is electrically coupled to the proximal side of the ultrasonic probe 100, and the other end is coupled to the ultrasonic diagnostic apparatus main body. A signal is transmitted to the ultrasound diagnostic apparatus main body via the cable 150, and a control signal from the ultrasound diagnostic apparatus main body is transmitted to the ultrasonic probe 100 via the cable 150.

Next, the inner structure of the ultrasonic probe 100 according to the present embodiment will be explained with reference to FIG. 2.

FIG. 2 is a cross-sectional drawing illustrating a part of the ultrasonic probe when being cut in a direction in which the piezoelectric elements are arranged in the ultrasonic probe 100 (i.e., an array direction).

The ultrasonic probe 100 shown in FIG. 2 includes a plurality of piezoelectric elements 201, a flexible print substrate 202, an intermediate layer 203, and a backing member 204. In the acoustic radiation direction (the +z direction in FIG. 2), structures commonly used in a typical ultrasonic probe, such as an acoustic matching layer and an acoustic lens, are added to the plurality of piezoelectric elements 201; however, the description thereof is omitted herein.

In the following, the flexible print substrate 202 may be called simply a substrate, or an FPC (flexible printed circuit) 202.

The plurality of piezoelectric elements 201 are acoustic/electric reversible sensing elements, such as a piezoelectric ceramics, etc. The plurality of piezoelectric elements 201 are arranged at a predetermined pitch.

The FPC 202 is arranged on the back surface of the plurality of piezoelectric elements 201, which is a surface opposite to the surface of the acoustic radiation side. The FPC 202 includes signal lines for signal transmission with each of the plurality of piezoelectric elements 201. As a wiring pattern, the signal lines are formed on the FPC 202 or inside of the FPC 202, so that a signal of each piezoelectric element 201 is transmitted to the proximal end side of the housing 101. The FPC 202 includes a region which is cut halfway through in a direction of the thickness of the FPC 202 at a predetermined pitch in such a manner that at least the signal lines are not cut (in FIG. 2, the region is shown as a region that is surrounded by a broken line and partially not separated).

In the example shown in FIG. 2, the FPC 202 according to the present embodiment has a two-layer structure which includes a first layer 202-1, which is a metal layer separated at the same pitch as the pitch for arranging the plurality of piezoelectric elements 201 to serve as an electrode for each of the plurality of piezoelectric elements 201, and a second layer 202-2 that is arranged on the back surface of the first layer 202-1 and in which a signal is transmitted from the electrodes to the proximal end side of the housing 101.

The intermediate layer 203 is a layer arranged on the back surface of the FPC 202 (more specifically, the second layer 202-2), and similar to the plurality of piezoelectric elements 201, is formed by arranging a plurality of layer members 203-1 at a predetermined pitch in an array direction. As a result, in the intermediate layer 203, a gap 203-2 is formed between adjacent layer members 203-1.

Specifically, in the intermediate layer 203, a plurality of layer members 203-1 are arranged in the array direction in such a manner that their positions respectively match the positions of the plurality of piezoelectric elements 201; as a result, gaps 203-2 are formed in such a manner that the location of the gap matches a space between the adjacent piezoelectric elements 201. The gap 203-2 may be filled with air or with a resin. The layer member 203-1 may be made of the same material as the material of the FPC 202, or the same material as that of the backing member 204.

The backing member 204 is arranged at the back surface of the intermediate layer 203 to suppress unnecessary vibration by absorbing ultrasonic waves transmitted to the proximal end side of the housing 101.

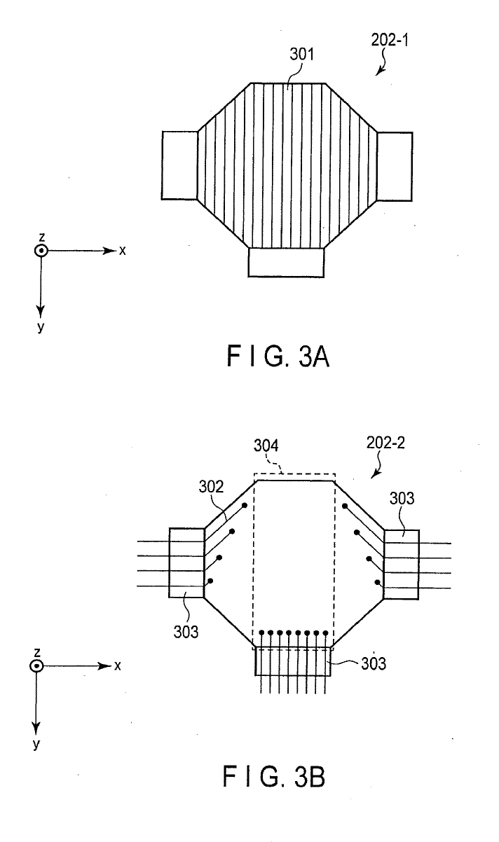

Next, an example of the wiring pattern of the FPC 202 will be described in detail with reference to FIG. 3A and FIG. 3B. FIG. 3A shows the first layer 202-1 of the FPC 202, and FIG. 3B shows the second layer 202-2 of the FPC 202.

As shown in FIG. 3A, the first layer 202-1 including a metal layer is divided at the predetermined pitch, that is, the same pitch as the pitch for arranging the plurality of piezoelectric elements 201 (the pitch is represented by each of the solid lines in FIG. 3A). Electrodes 301 of the plurality of the piezoelectric elements 201 are thereby formed.

As shown in FIG. 3B, to transmit a signal from each electrode 301 of the first layer 202-1 to the proximal end side, the wiring pattern is formed in the second layer 202-2 so as to extract each signal line 302 to the extraction region 303. In other words, when seen from the direction of the thickness of the FPC 202, the wiring pattern is extracted in a plurality of directions so as to cross over a plurality of piezoelectric elements 201 in an array direction of the piezoelectric elements. Each signal line 302 is electrically connected to each electrode 301 of the first layer 202-1 by the through holes formed in the second layer 202-2, and the signal line 302 is extracted to the extraction region 303 as a channel independent from the piezoelectric element 201 to which each electrode 301 is joined.

Since there are three extraction regions 303 in the example shown in FIG. 3B, the signal lines 302 other than the signal lines 302 existing in the center region 304 of the second layer 202-2 are extended and extracted, crossing over the plurality of piezoelectric elements, when seen from the z-axis direction. According to the ultrasonic probe 100 of the present embodiment, as shown in FIG. 2, the FPC 202 includes a region which is cut halfway through at a predetermined pitch in the direction of the thickness of the FPC 202, so that at least the signal lines are not cut. Accordingly, a space is formed between the adjacent electrodes 301 in the first layer 202-1, whereas the signal lines 302 are not cut (not separated) in the second layer 202-2.

The signal lines 302 in the center region 304 are extracted to the extraction region 303, without crossing over the plurality of piezoelectric elements along the array direction, when seen from the z-axis direction. Accordingly, in the center region 304 of the second layer 202-2 where the wiring pattern is not extended in the array direction, the second layer 202-2 is completely separable in the direction of the thickness of the second layer 202-2 at a predetermined pitch, without cutting the signal lines 302. Thus, only the resin layer in the second layer 202-2 of the FPC is separated, whereas the wiring pattern is not cut.

Next, an example of a method of manufacturing the inner structure of the ultrasonic probe 100 according to the present embodiment shown in FIG. 2 will be explained.

In step 1, the FPC and the intermediate layer 203 are joined with adhesive glue. Herein, a thin backing member is assumed as an intermediate layer 203.

In step 2, dicing is carried out from the back surface side of the thin backing member, avoiding cutting the FPC. By this dicing, an arrangement of a plurality of layer members (the backing members, in this example) can be formed.

In step 3, a piezoelectric body having a size approximately the same as the FPC is joined to the acoustic radiation side (a surface facing the surface to which a thin backing member is joined) of the FPC.

In step 4, the piezoelectric body is diced halfway through the FPC from the acoustic radiation side, in other words, diced up to a position where the FPC is not completely separated, at a same pitch as the pitch of dicing the thin backing member in step 2. A plurality of piezoelectric elements 201 are thereby formed, and the separated portions in the FPC are formed as the first layer 202-1, i.e., the electrodes 301. When using a pre-joint of the first layer 202-1 with the second layer 202-2 used as the FPC 202, only the first layer 202-1 is separated, and the second layer 202-2 is left unseparated.

In step 5, a thick backing member 204 is joined to the back surface of the thin backing member with adhesive glue, such as an epoxy resin. Thus, both of the piezoelectric elements 201 and the backing member are cut halfway through at the same pitch, without separating the FPC in the acoustic radiation direction (the z-axis direction). To keep the thickness of the adhesive glue accurate, a sheet-type adhesive glue may be used. The steps of manufacturing the inner structure of the ultrasonic probe 100 are completed.

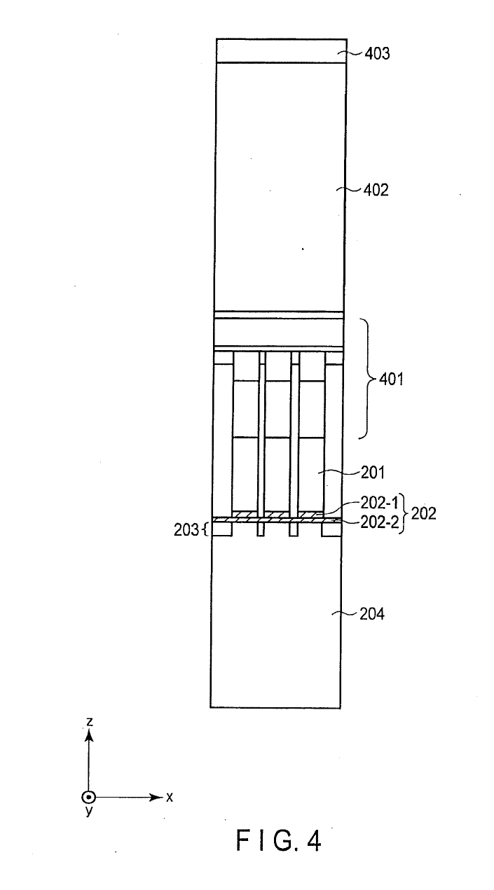

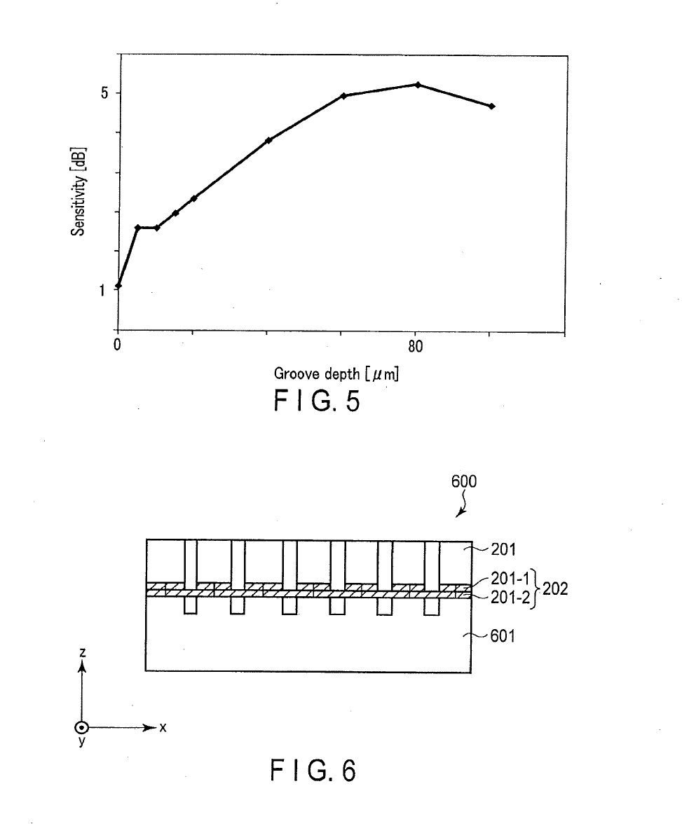

Next, a simulation result of the ultrasonic probe according to the present embodiment will be explained with reference to FIGS. 4 and 5.

FIG. 4 is a drawing showing a computation model for the inner structure of the ultrasonic probe according to the present embodiment for simulation using a finite element method (FEM). According to the computation model, acoustic matching layers 401 are stacked on the acoustic radiation side of the piezoelectric elements 201, and an acoustic lens 402 is stacked on the acoustic radiation side of the acoustic matching layers 401. In simulation, the acoustic lens 402 is in contact with water 403, so that the layer of water 403 is assumed as a transmission sound pressure measuring position.

FIG. 5 is a drawing showing a simulation result based on the computation model shown in FIG. 4. The vertical axis indicates transmission sensitivity [dB] of a transmission sound pressure spectrum, and the horizontal axis indicates a depth [μm] of a gap 203-2 in the intermediate layer 203 (a depth with respect to the side opposite to the acoustic radiation side). The central frequency used in the ultrasonic probe in the simulation is approximately 5 MHz.

As shown in FIG. 5, in the simulation model where the depth of the gap 203-2 is approximately 80 μm, if the computation model does not have the intermediate layer 203, in other words, in comparison to a computation model where a backing model with no gap 203-2 is arranged, transmission sensitivity is improved by approximately 5 dB.

(First Modification)

A first modification of the ultrasonic probe 100 according to the present embodiment will be explained with reference to FIG. 6.

FIG. 6 is a drawing showing an inner structure of an ultrasonic probe 600 according to the first modification. The ultrasonic probe 600 includes a plurality of piezoelectric elements 201, an FPC 202, and a backing member 601.

In the backing member 601, a groove (gap) is formed at the same position as the pitch of the piezoelectric elements 201 in the array direction. The backing member 601 is arranged at the back surface of the FPC 202 by being joined with adhesive glue. As shown in FIG. 6, instead of the backing member 204 joined to and arranged on the back surface of the intermediate layer 203, a groove may be directly formed in the backing member. Thus, the intermediate layer 203 and the backing member 204 can be integrally formed.

(Second Modification)

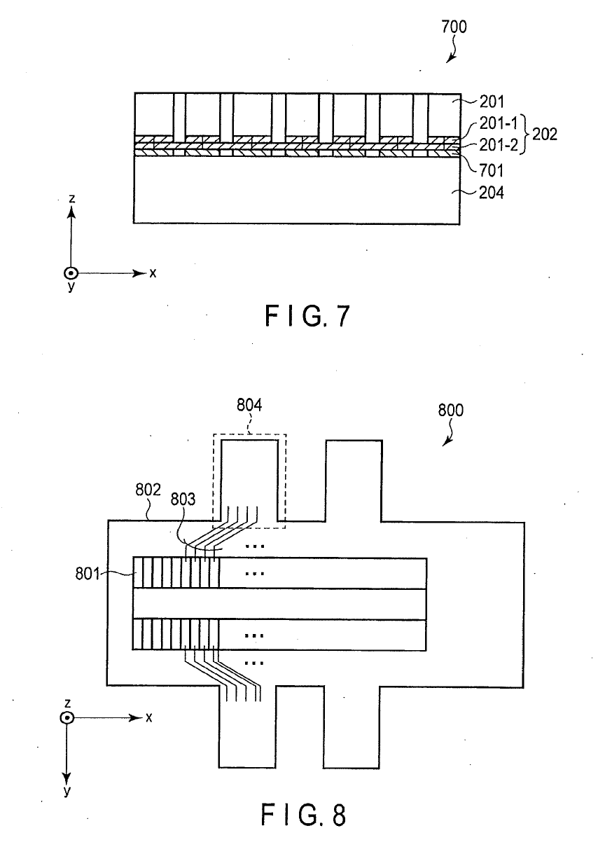

A second modification of the ultrasonic probe 100 according to the present embodiment will be explained with reference to FIG. 7.

FIG. 7 is a drawing showing an inner structure of an ultrasonic probe 700 according to the second modification. The ultrasonic probe 700 includes a plurality of piezoelectric elements 201, the first layer 202-1 of the FPC, the second layer 202-2 of the FPC, a third layer 701 of the FPC, and the backing member 204.

In other words, the ultrasonic probe 700 according to the second modification is an example where the intermediate layer 203 is made of the same material as the FPC.

The backing member 601 according to the first modification may be arranged in the back surface of the third layer 701 of the FPC, instead of the backing member 204.

An intracavitary probe, such as a so-called bar hole probe, is assumed as the above-described ultrasonic probe; however, an ultrasonic probe of the 1.5-dimensional array-type (a so-called 1.5-dimensional array ultrasonic probe) is also similarly applicable.

An example of a wiring pattern for an FPC in the 1.5-dimensional array ultrasonic probe will be explained with reference to FIG. 8.

In a 1.5-dimensional array ultrasonic probe, piezoelectric elements arranged as a one-dimensional array are also arranged in the slice direction; accordingly, from the viewpoint of arrangement of two-dimensional piezoelectric elements, a 1.5-dimensional array ultrasonic probe has a structure similar to a two-dimensional array ultrasonic probe in which piezoelectric elements are arranged in a matrix. On the other hand, since so-called two-dimensional scanning, which moves ultrasonic beam in an azimuth direction, is applied to collect two-dimensional tomographic image data, a 1.5-dimensional array ultrasonic probe has a structure more similar to a one-dimensional array ultrasonic probe than to a two-dimensional array ultrasonic probe which can collect three-dimensional volume data. Thus, since the probe is in-between a one-dimensional ultrasonic probe and a two-dimensional ultrasonic probe, it is called a 1.5-dimensional array ultrasonic probe.

FIG. 8 is a perspective view of the 1.5-dimensional array ultrasonic probe viewed from the acoustic radiation side. The 1.5-dimensional array ultrasonic probe includes a plurality of piezoelectric elements 801, an FPC 802 arranged on the back surface of the piezoelectric elements 801, and a wiring pattern 803 of signal lines for signal transmission with the piezoelectric elements 801 arranged in the FPC 802.

The plurality of piezoelectric elements 801 are arranged at a predetermined pitch in the array direction. The wiring pattern 803 is laid crossing a plurality of the piezoelectric elements 801 in the array direction to transmit a signal to the extraction region 804. Therefore, similar to the above-described cases, the FPC 802 is cut halfway through at a predetermined pitch in the direction of the thickness of the FPC 802 for the region where the wiring pattern crosses the arrangement of the plurality of piezoelectric elements 801 in the array direction when seen from the z-axis direction, without cutting at least the signal lines.

(Third Modification)

In the above-described embodiment, the FPC 202 is explained as a structure consisting of the first layer 202-1 which serves as the electrode 301 and the second layer 202-2 on which a wiring pattern is formed, and the layers are separately explained; however, the substrate 202 may be a structure that is not divided into the first layer 202-1 and the second layer 202-2.

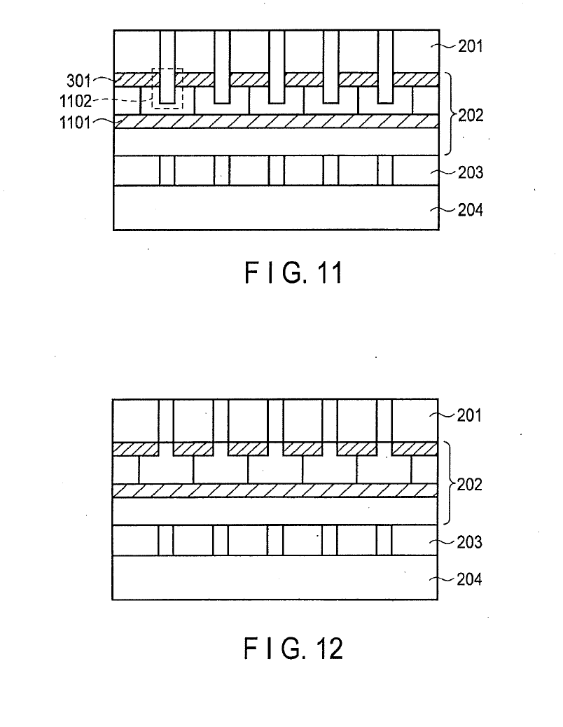

The third modification of the ultrasonic probe 100 according to the present embodiment will be explained with reference to FIG. 11.

As shown in FIG. 11, in the FPC 202, an electrode 301 is formed on a surface to which a plurality of piezoelectric elements 201 are connected. In the inside of the FPC 202, a wiring pattern 1101, which is a signal line to be electrically connected to the electrode 301 via through holes.

The FPC 202 may be cut halfway through at a predetermined pitch up to a position where the wiring pattern is not cut off, as shown in the region 1102. In other words, if a plurality of piezoelectric elements 201 are formed by dicing a piezoelectric body at a predetermined pitch, it is allowed to cut the FPC 202 up to a position as indicated by the region 1102.

(Fourth Modification)

The fourth modification of the ultrasonic probe 100 according to the present embodiment will be explained with reference to FIG. 12.

The fourth modification shows an example where the FPC 202 is not cut. If the electrodes 301 for a plurality of piezoelectric elements 201 are formed on the FPC 202 at a predetermined pitch, the FPC 202 is not necessarily cut.

In the above-described embodiment, an example of the FPC 202 having a two-layer structure was explained; however, the FPC 202 is not limited thereto, and it may have a single-layer structure or a three-layer structure. For example, if the FPC 202 has a structure of three or more layers, the first layer of the FPC 202 serves as an electrode of the piezoelectric elements, and the single lines of the second layer and thereafter should not be cut.

According to the present embodiment and the modifications thereof as described above, it is possible to provide a gap between the FPC and the backing member by the intermediate layer even when a wiring pattern requiring wiring density for signal transmission is used, thereby providing a gap on the back surface side of the FPC without cutting the wiring pattern and improving ultrasonic transmission/reception sensitivity in an ultrasonic probe.

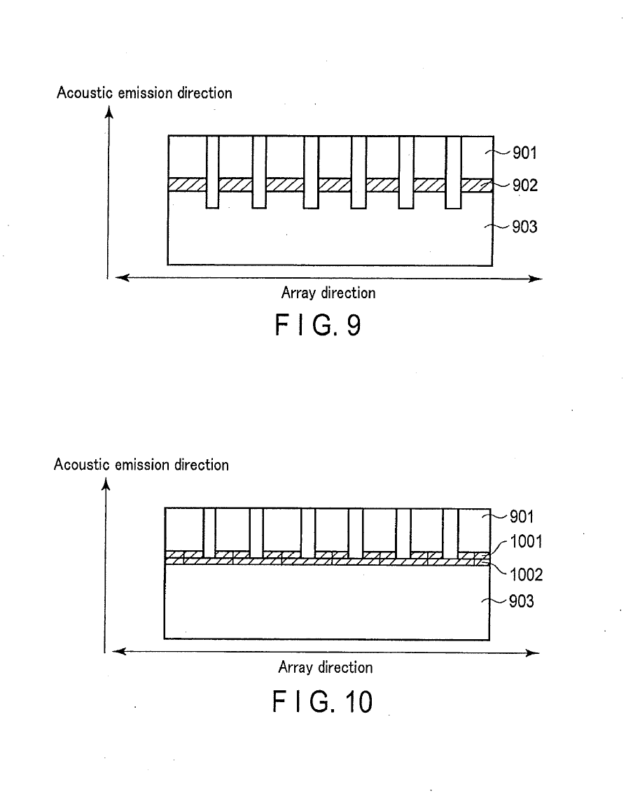

In the following, a configuration of a conventional ultrasonic probe will be explained with reference to FIG. 9 to FIG. 10.

A conventional ultrasonic probe consists of a piezoelectric body 901, an FPC 902, and a backing member 903. Generally, separating the piezoelectric body 901 is carried out by dicing, and the piezoelectric body is cut through to the FPC 902 and the backing member 903 from the acoustic radiation side.

However, if wiring density of an FPC is required, the wiring needs to be continuous in an array direction, without cutting a wiring pattern in the FPC. Thus, dicing as shown in FIG. 9 cannot be carried out.

On the other hand, FIG. 10 shows a state where dicing is carried out on the FPC halfway through without cutting the wiring pattern, and there are a separated FPC 1001 and an unseparated FPC 1002. Since dicing is not carried out up to the backing member 903 and a gap is not formed in the backing member 903 as shown in FIG. 10, there is a problem of degraded sensitivity in ultrasonic transmission and reception.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic probe comprising:
a plurality of piezoelectric elements that are arranged at a predetermined pitch;
a substrate that is arranged on a back surface of the plurality of piezoelectric elements and includes signal lines for signal transmission with the plurality of piezoelectric elements and a wiring pattern for extracting a signal outside of the ultrasonic probe;
an intermediate layer that is arranged on a back surface of the substrate and in which a plurality of layer members are arranged in an array direction of the piezoelectric elements at the predetermined pitch; and
a backing member that is arranged on a back surface of the intermediate layer.

2. The probe according to claim 1, wherein the layer member is made of a same material as a material of the backing member.

3. The probe according to claim 1, wherein the layer member is made of a same material as a material of the substrate.

4. The probe according to claim 1, wherein the substrate includes:
a first layer that is joined to the back surface of the plurality of piezoelectric elements and separated at the predetermined pitch as electrodes of the piezoelectric elements; and
a second layer that is arranged on a back surface of the first layer and on which a wiring pattern related to the signal lines which are electrically connected to the electrodes is formed.

5. The probe according to claim 4, wherein in the second layer, a region in which the wiring pattern is not extended in an array direction is separable in a direction of a thickness of the substrate.

6. The probe according to claim 1, wherein the substrate includes a region that is cut halfway through at the predetermined pitch in a direction of a thickness of the substrate so that at least the signal lines are not cut.

7. The probe according to claim 1, wherein the wiring pattern is extracted in a plurality of directions so as to cross over a plurality of piezoelectric elements in the array direction, when seen from a direction of a thickness of the substrate.

8. The probe according to claim 1, wherein the probe is an intracavitary probe.
